(19) United States
(12) Patent Application Publication
(10) Pub. No.: US 2022/0370538 A1
(43) Pub. Date: Nov. 24, 2022

(54) METHOD FOR EXTRACTING FUNCTIONAL INGREDIENTS OF MULBERRY LEAVES USING ENZYMES

(71) Applicant: Shanxi Agricultural University, Jinzhong City (CN)

(72) Inventors: Lin Hao, Taiyuan City (CN); Qianqian Li, Liaocheng City (CN); Liping Hao, Taiyuan City (CN)

(21) Appl. No.: 17/551,221

(22) Filed: Dec. 15, 2021

(30) Foreign Application Priority Data
May 24, 2021 (CN) 2021105633570

(51) Int. Cl. A61K 36/605; C12P 1/02; C12P 21/02; A23L 33/105

(52) U.S. Cl. CPC A61K 36/605; C12P 1/02; C12P 21/02; A23L 33/105; A61K 2236/19; A61K 2236/51; A61K 2236/53; A23V 2002/00

(57) ABSTRACT

A method for extracting functional ingredients of mulberry leaves using enzyme is provided. A method for preparing crude cellulase enzyme solution and crude pectinase enzyme solution includes: preparing PDA slant culture mediums; preparing fermentation mediums for producing cellulase and pectinase; preparing solid bacteria; fermentation; preparing crude cellulase enzyme solution and crude pectinase enzyme solution. A preparation method of mulberry leaf concentrate is provided and includes: enzymatic hydrolysis, enzyme inactivation and concentration. A method for preparing mulberry leaf products from mulberry leaf concentrate is provided. Cellulase and pectinase prepared by the stock culture of agaric are safer and more reliable; the content and yield of protein and other functional ingredients in mulberry leaves are improved; the mulberry leaf product can be directly applied to processing and production of food, health care products and cosmetics which improves comprehensive utilization rate of mulberry leaf raw materials.

### TECHNICAL FIELD

The disclosure relates to the technical field of enzymes, in particular to a method for extracting functional ingredients of mulberry leaves (also referred to as Folium Mori) using enzymes.

### DESCRIPTION OF RELATED ART

Mulberry leaves (also referred to as Folium Mori) may be both medicinal and edible. It is recorded in New Compilation of Materia Medica that the efficacy of mulberry leaves is better than cortex mori. Mulberry leaf is best at replenishing nutrients to bone marrow, invigorating the essence in the kidneys, treating night sweats, activating the brain, brightening the eyes, promoting blood circulation and generating fluids, calming the fetus, harmonizing blood vessels, and facilitating movements of joints. In recent years, research on mulberry leaves has revealed that mulberry leaves are rich in many functional ingredients, such as polysaccharides, alkaloids and flavonoids. Mulberry leaves have various functions such as lowering blood sugar, blood pressure and blood lipids, enhancing immunity, removing melanin, and whitening the skin. As a medicinal and food plant, mulberry leaves have outstanding health care functions. However, the exploitation is very limited at present. For example, Zhu Tianming et al. published the article titled as “Process of cellulase assisted extraction of leaf protein from mulberry leaves”, which studied the extraction of protein from mulberry leaves using cellulase assisted extraction, and the extraction rate of mulberry leaf protein was only 6.5 g of protein per 100 g of mulberry leaves. Therefore, a new extraction process needs to be developed to improve the content and yield of functional ingredients in mulberry leaf products.

Meanwhile, the cellulase and pectinase used in the prior art for the extraction of mulberry leaf components mainly come from fungi, and the more typical ones are Xylophaga, Aspergillus and Penicillium. Inevitably, the process of fermentation and production of enzymes by Aspergillus and Penicillium is accompanied by the production of toxins. Even Aspergillus with high safety often produces trace uncertain harmful substances in the process of enzyme production, and the later separation and detoxification process is complex and difficult; at the same time, the slurry obtained by enzyme treatment of mulberry leaves in the later stage needs to go through the concentration process, so that even a small amount of harmful substances will increase the content due to concentration. Therefore, how to ensure the edible safety of cellulase and pectinase used in food and drug production is an urgent technical problem to be solved.

### DETAILED DESCRIPTION OF THE EMBODIMENTS

The purpose of the disclosure is to provide a method for extracting functional ingredients of mulberry leaves with enzymes, so as to solve the problems existing in the prior art. On the one hand, the whole preparation process of cellulase and pectinase does not produce any toxins and harmful substances, which is safer and more reliable and ensures the safety of people's consumption; on the other hand, using cellulase and pectinase to extract the functional ingredients from mulberry leaves may improve the content and yield of functional ingredients in mulberry leaves.

In order to achieve the above purpose, the disclosure provides the following scheme:

one of the objects of the disclosure is to provide a method for preparing crude cellulase enzyme solution (also referred to as crude enzyme solution of cellulase), including:

S1: preparing the PDA slant culture medium;

S2: preparing of cellulase-producing fermentation medium, including: mixing bran, orange peel powder, ammonium sulfate, magnesium sulfate, potassium hydrogen phosphate and water evenly and sterilizing to obtain cellulase-producing fermentation medium;

S3: preparing of solid bacteria (also referred to as solid strain), including: taking the stock culture of Auricularia auricula (also referred to as agaric) into PDA slant culture medium for culture to obtain mycelium;

S4: fermentation, including: taking the mycelium into the cellulase-producing fermentation medium, mixing well and culturing; and

S5: preparing of crude cellulase enzyme solution, including: taking the cellulase-producing fermentation medium cultured in S4, adding water, shaking, standing still, filtering and centrifuging to obtain crude cellulase enzyme solution.

Further, the pH value of a mixture of bran, straw powder, ammonium sulfate, potassium dihydrogen phosphate, magnesium sulfate, calcium chloride and water mixed in S2 is 3.5-5.

Further, in S2, the mass ratio of bran, straw powder, ammonium sulfate, potassium dihydrogen phosphate, magnesium sulfate, calcium chloride and water is 562.5:437.5:40:3:1:1:1500.

Further, the temperature of the culturing in S4 is 25-32° C., and the duration is 3-4 days.

The second object of the disclosure is to provide a method for preparing crude pectinase enzyme solution (also referred to as crude enzyme solution of pectinase), including:

S1: preparing the PDA slant culture medium;

S2: preparing of pectinase-producing fermentation medium, including: mixing bran, orange peel powder, ammonium sulfate, magnesium sulfate, potassium hydrogen phosphate and water evenly and sterilizing to obtain pectinase-producing fermentation medium;

S3: preparing of solid bacteria, including: taking the stock culture of agaric into PDA slant culture medium for culture to obtain mycelium;

S4: fermentation, including: taking the mycelium into the pectinase producing fermentation medium, mixing well and culturing;

S5: preparing of crude pectinase enzyme solution, including: taking the pectinase producing fermentation medium cultured in S4, adding water, shake, standing still, filtering and centrifuging to obtain crude pectinase enzyme solution.

Further, the pH value of a mixture of bran, orange peel powder, ammonium sulfate, magnesium sulfate, dipotassium hydrogen phosphate and water mixed in S2 is 4-5.

Further, in S2, the mass ratio of bran, orange peel powder, ammonium sulfate, magnesium sulfate, dipotassium hydrogen phosphate and water is 2000:264:46:1:3:1270.

Further, the temperature of the culturing in S4 is 25-32° C. and the duration is 3-4 days.

The third object of the disclosure is to provide a preparation method of mulberry leaf concentrate, including:

S1: mixing the mulberry leaves with water and mashing to prepare slurry, and then adding the crude cellulase enzyme solution mentioned above and the crude pectinase enzyme solution mentioned above for enzymatic hydrolysis to obtain a target slurry;

S2: subjecting the target slurry after the enzymatic hydrolysis in S1 to enzyme killing treatment, and then concentrating to obtain mulberry leaf concentrate.

Further, the preparation method of mulberry leaf concentrate further includes: filtering the slurry before the S2.

Further, the mass ratio of the crude cellulase enzyme solution to the crude pectinase enzyme solution is 3:2.

The fourth object of the disclosure is to provide a mulberry leaf product, including the mulberry leaf concentrate prepared by the above preparation method; the dosage form of the mulberry leaf product is dropping pills, powder or tablets.

The disclosure discloses the following technical effects:

(1) The crude cellulase enzyme solution and the crude pectinase enzyme solution in this disclosure are prepared by fermentation of the stock culture of agaric, and the whole preparation process is safe and toxin-free (agaric, as an edible fungus, its safety has been verified for hundreds of years), and the crude cellulase enzyme solution and the crude pectinase enzyme solution prepared are safe and reliable to ensure the safety of consumption, which is obviously better than those cellulase and pectinase produced by Penicillium, Trichoderma, Aspergillus niger, etc. in the current market.

(2) The disclosure uses orange peel powder as an inducer in the pectinase producing fermentation medium to induce agaric mycelium to produce pectinase (pectinase belongs to inducible enzyme, and the existence of inducer can obviously induce the transcription and expression of the corresponding enzyme gene);

(3) The disclosure uses the crude enzyme solution of cellulase and pectinase prepared by fermentation of the stock culture of agaric to extract functional ingredients from mulberry leaves, which improves the content and yield of protein and other functional ingredients in mulberry leaves while ensuring the safety of the preparation process;

(4) The mulberry leaf concentrate prepared by the disclosure has strong functional activity. By making the mulberry leaf concentrate into dropping pills, powder or tablets, it can be used as auxiliary materials or directly applied to the processing and production of food, health products (anti-oxidation, hypoglycemic, blood pressure reduction, etc.) and cosmetics (inhibiting the activity of tyrosinase, thereby inhibiting the formation of melanin in the body), so as to improve the comprehensive utilization rate of mulberry leaf raw materials. The market potential is huge and has good economic and social benefits.

### BRIEF DESCRIPTION OF DRAWINGS

In order to illustrate more clearly the technical solutions in the embodiments of the disclosure or in the prior art, a brief description of the drawings to be used in the embodiments will be given below, and it will be obvious that the drawings in the following description are only some of the embodiments of the disclosure, and that other drawings can be obtained on the basis of these drawings without creative work by a person of ordinary skill in the art.

FIG. 1 shows the effect of fermentation time on cellulase and pectinase activities produced by the strain;

FIG. 2 shows the effect of fermentation temperature on cellulase and pectinase activities produced by the strain;

FIG. 3 shows the effect of fermentation pH on cellulase and pectinase activities produced by strains;

FIG. 4 shows the effect of the addition amount of crude cellulase enzyme solution and crude pectinase enzyme solution on the content of total flavonoids in mulberry leaves;

FIG. 5 shows the effect of the ratio of crude cellulase enzyme solution and crude pectinase enzyme solution on the content of total flavonoids in mulberry leaves;

FIG. 6 shows the effect of pH value on the content of total flavonoids in mulberry leaves;

FIG. 7 shows the effect of extraction temperature on the content of total flavonoids in mulberry leaves;

FIG. 8 shows the effect of extraction time on the content of total flavonoids in mulberry leaves.

### DETAILED DESCRIPTION OF THE EMBODIMENTS

Various exemplary embodiments of the disclosure are now described in detail, and this detailed description should not be considered a limitation of the disclosure, but should be understood as a more detailed description of certain aspects, features and embodiments of the disclosure.

It is to be understood that the terms described in the disclosure are intended to describe particular embodiments only and are not intended to limit the disclosure. Further, with respect to the range of values in the disclosure, it is to be understood that each intermediate value between the upper and lower limits of the range is also specifically disclosed. Each smaller range between any stated value or intermediate value within a stated range and any other stated value or intermediate value within a stated range is also included in the disclosure. The upper and lower limits of these smaller ranges may be independently included or excluded from the scope.

Unless otherwise indicated, all technical and scientific terms used herein have the same meaning as commonly understood by those of ordinary skill in the art described in the disclosure. Although the disclosure describes only preferred methods and materials, any methods and materials similar or equivalent to those described herein may also be used in the implementation or testing of the disclosure. All literature referred to in this specification is incorporated by reference for the purpose of disclosing and describing the methods and/or materials associated with said literature. In the event of conflict with any incorporated literature, the contents of this specification shall prevail.

Various improvements and variations may be made to specific embodiments of the specification of the disclosure without departing from the scope or spirit of the disclosure, as will be apparent to those skilled in the art. Other embodiments obtained from the specification of the disclosure will be apparent to those skilled in the art. The specification and embodiments of the disclosure are exemplary only.

The terms “contains”, “includes”, “has”, “contains”, etc. used herein are open-ended terms, i.e., meaning including but not limited to.

The agaric (also referred to as Auricularia auricula) used in this disclosure is white back fungus (also referred to as Auricularia polytricha), which was purchased from Wuhan Zhouyulin Edible Fungi Research Institute.

Embodiments of the disclosure are performed under aseptic conditions, unless otherwise specified.

### Embodiment 1, Preparing of Crude Cellulase Enzyme Solution

S1: preparing of PDA slant culture medium, including: weighing 200 g of peeled potatoes, cutting the potatoes into small squares of about 1 cm, pouring the squares into a ceramic jar, adding 800 mL of water, boiling for 15 min, filtering the boiling solution into a beaker with two layers of gauze, then adding water to 1 L, pouring the filtrate into a cleaned porcelain jar again, adding 20 g of glucose and 20 g of agar, and stirring with a glass rod until the materials are fully dissolved; pouring the above liquid into a 1 L beaker, adding water to 1 L, then subpackaging the liquid into 18 (mm)×180 (mm) test tubes, putting a cotton plug or rubber plug on the test tube mouth after subpackage, wrapping with newspaper, putting the tubes in an autoclave, sterilizing at 121° C. for 20 minutes, after sterilization, putting the tubes on a slant while it is hot, and obtain the PDA slant culture medium for later use after solidification;

S2: preparing of cellulase-producing fermentation medium, including: accurately weighing 4.5 g of bran, 3.5 g of corn straw powder, 0.32 g of ammonium sulfate, 0.024 g of potassium dihydrogen phosphate, 0.008 g of magnesium sulfate, 0.008 g of calcium chloride, putting the above materials into a 250 mL triangular flask, adding 12 mL of distilled water with initial pH of 4, stirring the solution well with a glass rod, then wrapping the conical bottle mouth with 8 layers of gauze, and wrapping two more layers of newspaper, putting the flask into the autoclave, sterilizing at 121° C. for 30 min, and cooling for standby;

S3: preparing of solid strain, including: introducing the white back fungus stock culture into PDA slant culture medium prepared in S1 under aseptic operation, then putting the medium in an incubator, cultivating at 30° C. for 7 days, and putting the medium with good growth state and no contamination by foreign bacteria into a refrigerator, and storing the medium at 4° C. for later use.

S4: fermentation, including: introducing the strain in PDA slant culture medium in S3 with sterilized inoculation hook, taking about 0.5 cm² mycelium into cellulase-producing fermentation culture medium, and mixing well, and culturing at 30° C. for 4 days, so as to ensure that there is no contamination by foreign bacteria during the culture process, which is also the guarantee of product safety;

S5: preparing of crude cellulase enzyme solution, including: weighing 10 g of cultured fermentation medium for cellulase production, adding 50 mL of distilled water, shaking for 10 min on a shaking table at 200 r/min, stand for 2h, filtering with four layers of gauze, centrifuging the filtrate at 3500 r/min for 10 min, and obtain supernatant as the crude cellulase enzyme solution.

Result: the enzyme activity of crude cellulase enzyme solution is 157.35 U/mL.

### Embodiment 2, Preparing of Crude Cellulase Enzyme Solution

Different from Embodiment 1, the initial pH value in S2 is 3.5, the culture temperature is 28° C., and the duration is 3.5 days.

Result: the enzyme activity of crude cellulase enzyme solution is 145.36 U/mL.

### Embodiment 3, Preparing of Crude Cellulase Enzyme Solution

Different from Embodiment 1, the initial pH value in S2 is 5, the culture temperature is 32° C., and the duration is 4 days.

Result: the enzyme activity of the crude cellulase enzyme solution is 140.27 U/ml.

### Embodiment 4, Preparing of Crude Pectinase Enzyme Solution

51: preparing of PDA slant culture medium, including: weighing 200 g of peeled potatoes, cutting the potatoes into small squares of about 1 cm, pouring the squares into a ceramic jar, adding 800 mL of water, boiling for 15 min, filtering the boiling solution into a beaker with two layers of gauze, then adding water to 1 L, pouring the filtrate into a cleaned porcelain jar again, adding 20 g of glucose and 20 g of agar, and stirring with a glass rod until the materials are fully dissolved; pouring the above liquid into a 1 L beaker, adding water to 1 L, then subpackaging the liquid into 18 (mm)×180 (mm) test tubes, putting a cotton plug or rubber plug on the test tube mouth after subpackage, wrapping with newspaper, putting the tubes in an autoclave, sterilizing at 121° C. for 20 minutes, after sterilization, putting the tubes on a slant while it is hot, and obtain the PDA slant culture medium for later use after solidification;

S2: preparing of pectinase-producing fermentation medium, including:
accurately weighing 10 g of bran, 1.32 g of orange peel powder, 0.23 g of ammonium sulfate, 0.005 g of magnesium sulfate and 0.015 g of dipotassium hydrogen phosphate, putting the above materials into a 250 mL triangular flask, adding 6.35 mL of distilled water with initial pH of 4, stirring the solution well with a glass rod, then wrapping the conical bottle mouth with 8 layers of gauze, and wrapping two more layers of newspaper, putting the flask into the autoclave, sterilizing at 121° C. for 30 min, and cooling for standby;

S3: preparing of solid strain, including: introducing the white back fungus stock culture into PDA slant culture medium prepared in S1 under aseptic operation, then putting the medium in an incubator, cultivating at 30° C. for 7 days, and putting the medium with good growth state and no contamination by foreign bacteria into a refrigerator, and storing the medium at 4° C. for later use;

S4: fermentation, including: introducing the strain in PDA slant culture medium in S3 with sterilized inoculation hook, taking about 0.5 cm² mycelium into cellulase-producing fermentation culture medium, and mixing well, and culturing at 30° C. for 4 days, so as to ensure that there is no contamination by foreign bacteria during the culture process, which is also the guarantee of product safety;

S5: preparing of crude pectinase enzyme solution, including: weighing 10 g of cultured fermentation medium for cellulase production, adding 50 mL of distilled water, shaking for 10 min on a shaking table at 200 r/min, stand for 2 h, filtering with four layers of gauze, centrifuging the filtrate at 3500 r/min for 10 min, and obtain supernatant as the crude pectinase enzyme solution.

Result: the enzyme activity of the crude pectinase enzyme solution is 101.77 U/ml.

### Embodiment 5, Preparing of Crude Pectinase Enzyme Solution

Different from Embodiment 4, the initial pH value in S2 is 3.5, the culture temperature is 28° C., and the duration is 3.5 days.

Result: the enzyme activity of crude pectinase enzyme solution is 98.43 U/mL.

### Embodiment 6, Preparing of Crude Pectinase Enzyme Solution

Different from Embodiment 4, the initial pH value in S2 is 5, the culture temperature is 32° C., and the time is 4 days.

Result: the enzyme activity of crude pectinase enzyme solution is 92.26 U/mL.

Agaric (grown by the differentiation of the mycelium of agaric) can be directly used as ingredients after processing and consumed in large quantities, while the mycelium of both Penicillium, Trichoderma, and Aspergillus cannot be directly used as ingredients for processing and consumption even in small quantities, which also proves that the safety of consumption of cellulase and pectinase produced by using agaric is guaranteed.

Effect of fermentation time on the enzymatic activity of cellulase and pectinase produced by the strain;

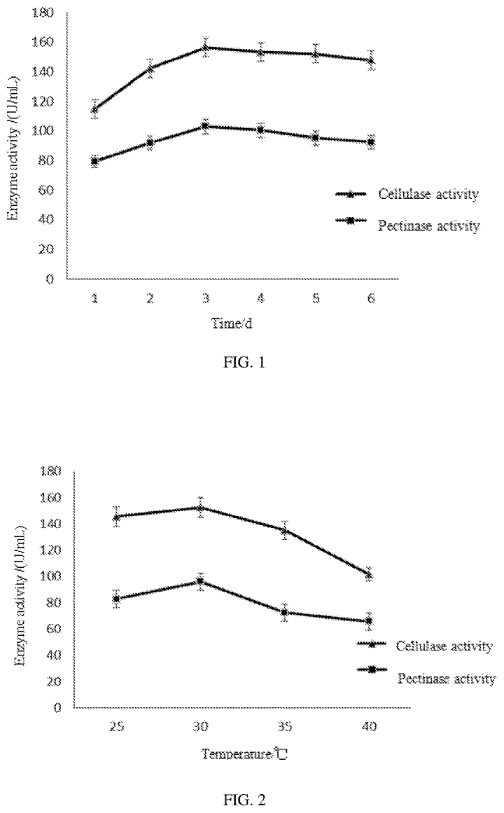

After the experiment, it is found that the cellulase enzyme activity and pectinase enzyme activity show a trend of increasing and then decreasing with the extension of fermentation time, and the highest cellulase enzyme activity and pectinase enzyme activity were 156.32 U/mL and 103.05 U/mL respectively at the third day. In the later stage of fermentation, the nutrients in the fermentation system are consumed, the harmful metabolites accumulated, and the enzyme production rate in the system slows down. In the late stage of fermentation, nutrient depletion and accumulation of harmful metabolites in the fermentation system slows down, some enzymes might be inactivated, and cellulase activity and pectinase activity shows a decreasing trend. The effect of fermentation time on the cellulase and pectinase enzyme activities of the strain is shown in FIG. 1.

The effect of fermentation temperature on the cellulase and pectinase activity of the strain.

This disclosure found that when the fermentation temperature is 30° C., the enzyme activity of cellulase and pectinase is the highest, 152.26 U/mL and 96.08 U/mL respectively, cellulase and pectinase are protein in nature, when the temperature is too high or too low, the enzyme activity will be reduced or even inactivated, so the enzyme activity of cellulase and pectinase gradually decreases with the increase of temperature. The effect of fermentation temperature on the enzymatic activity of cellulase and pectinase produced by the strain is shown in FIG. 2.

Effect of fermentation pH on the enzymatic activity of cellulase and pectinase produced by the strain.

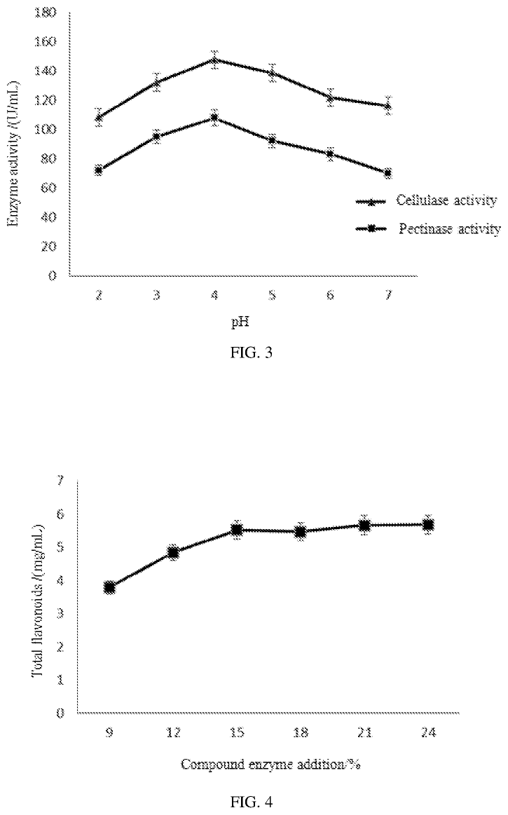

It is found that the cellulase and pectinase enzyme activities showed a trend of increasing and then decreasing with the increase of fermentation pH, and the highest cellulase and pectinase enzyme activities are 147.55 U/mL and 107.95 U/mL respectively when the initial pH of fermentation is 4. Too high or too low pH will also reduce the enzyme activities. The effect of fermentation pH on the cellulase and pectinase enzyme activities of the strains is shown in FIG. 3.

### Embodiment 7, Preparing of Mulberry Leaf Concentrate

S1: selecting fresh mulberry leaves from season to autumn without diseases and insect pests, cleaning the leaves with clean water, removing surface moisture, and removing leaf stalks, that is, taking 10 g mulberry leaves according to the material-water ratio of 1:7, putting them in a beater, adding 70 mL of water for beating for 2.5 min, and after beating, adding crude cellulase enzyme solution and crude pectinase enzyme solution prepared in Embodiment 1 and Embodiment 4 according to the mass ratio of 3:2, mixing the crude cellulase enzyme solution and crude pectinase enzyme solution according to the ratio of 3:2, accounting for 15% of the slurry mass obtained by mixing mulberry leaves and water for beating, and performing enzymolysis at pH 4.5 and temperature 50° C. for 2 h;

S2: filtering with double-layer 40 mesh filter cloth to obtain filtrate;

S3: putting the filtrate in a water bath at 80° C. to inactivate the enzyme for 15 min, then transferring the filtrate to a rotary evaporator, and concentrate to 10 mL at 60° C. and −0.1 MPa to obtain mulberry leaf concentrated solution.

### Embodiment 8, Preparing of Mulberry Leaf Concentrate

The difference from Embodiment 7 is that S2 is omitted.

Effect of addition of crude cellulase enzyme solution and crude pectinase enzyme solution on the total flavonoid content of mulberry leaves:

Experiments show that when the enzymolysis temperature is 50° C., the enzymolysis time is 2 h, the initial pH is 4.5, and the ratio of crude cellulase enzyme solution to crude pectinase enzyme solution is 3:2, the content of total flavonoids in mulberry leaves increases significantly with the increase of cellulase and pectinase addition, which indicates that cellulase and pectinase have the function of destroying plant cell walls, and the active components in cells after wall breaking are easier to release; when the addition of cellulase and pectinase is 15% of the slurry mass of mulberry leaves mixed with water, the content of total flavonoids is the highest, which is 5.52 mg/ml; when the addition of cellulase and pectinase is more than 15%, the content of total flavonoids has no obvious change. FIG. 4 shows the influence of the amount of crude cellulase enzyme solution and crude pectinase enzyme solution on the total flavonoids content in mulberry leaves.

Effect of the ratio of crude cellulase enzyme solution and crude pectinase enzyme solution on the total flavonoid content of mulberry leaves:

It is found that when the ratio of crude cellulase enzyme solution to crude pectinase enzyme solution is 3:2, the highest total flavonoid content of mulberry leaves is 5.232 mg/mL, indicating that cellulase and pectinase acted synergistically to destroy the cell wall of mulberry leaves, which facilitates the leaching of active ingredients from mulberry leaves. When the ratio of crude cellulase enzyme solution to crude pectinase enzyme solution is 0:5, the total flavonoid content of mulberry leaves is the lowest, indicating that when only pectinase is present, it is not very helpful for the leaching of active ingredients from mulberry leaves. The effect of the ratio of crude cellulase enzyme solution to crude pectinase enzyme solution on the total flavonoid content of mulberry leaves is shown in FIG. 5.

Effect of pH on the total flavonoid content of mulberry leaves:

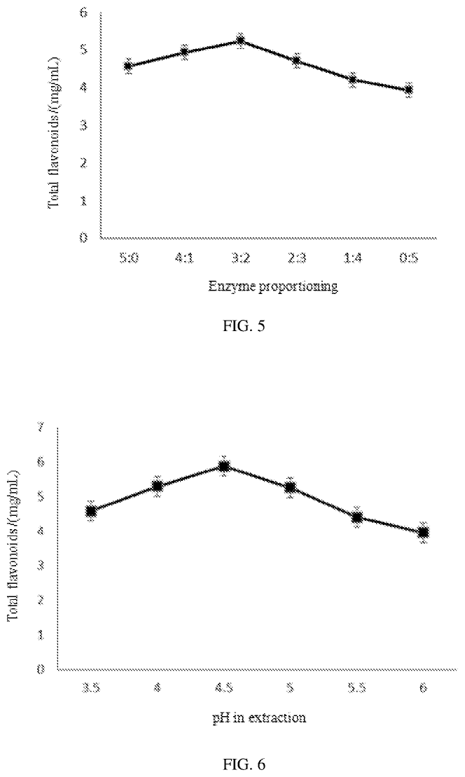

It is found that the total flavonoid content of mulberry leaves showed a trend of increasing and then decreasing with the increase of pH value, and when the pH is 4.5, the content of mulberry leaf flavonoid reached the maximum of 5.87 mg/mL, which is due to the fact that cellulase and pectinase functioned within a certain value, and the pH value is too low or too high to inhibit their activities, thus affecting the effect of cellulase and pectinase on the cell wall of mulberry leaves. The effect of pH on the total flavonoid content of mulberry leaves is shown in FIG. 6.

Effect of extraction temperature on the total flavonoid content of mulberry leaves.

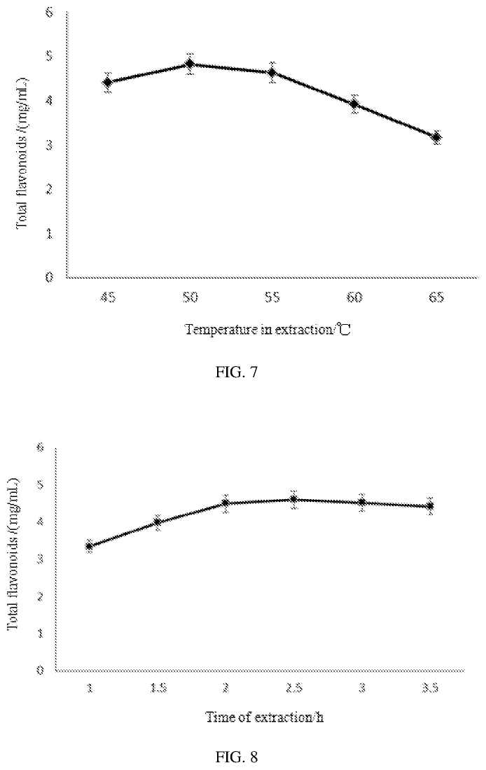

It is found that when the temperature is 50° C., the maximum content of mulberry leaf flavonoids is 4.827 mg/mL, while when the temperature exceeded 55° C., the content of mulberry leaf flavonoids shows a decreasing trend due to the high temperature, resulting in the partial inactivation of cellulase and pectinase. The effect of extraction temperature on the total flavonoid content of mulberry leaves is shown in FIG. 7.

Effect of extraction time on the total flavonoid content of mulberry leaves:

It is found after the test that the content of total flavonoids of mulberry leaves gradually increases with the increase of time, and when it is greater than 2 h, the content of total flavonoids of mulberry leaves does not increase significantly. The effect of extraction time on the total flavonoid content of mulberry leaves is shown in FIG. 8.

### Embodiment 9, Preparing of Mulberry Leaf Dropping Pills

A proper amount of medicinal polyethylene glycol 6000 and medicinal polyethylene glycol 4000 are put in a beaker according to the ratio of 1:1, and heated to a liquid state in a water bath; adding the mulberry leaf concentrated solution prepared in Embodiment 7 according to the ratio of matrix to mulberry leaf concentrated solution of 2:1, stirring and mixing evenly, and standing in a water bath for 20 minutes to eliminate bubbles; at 80° C., dropping the concentrated solution into dimethyl silicone oil, wherein the dropping distance is 5 cm and the temperature of dimethyl silicone oil is 5° C.; cooling the concentrated solution to make pills, wiping off dimethyl silicone oil attached to the surface of mulberry drop pills, and drying at room temperature (25° C.) to obtain mulberry dropping pills with excellent shape, size and color.

### Embodiment 10, Preparing of Mulberry Leaf Powder

Drying the mulberry leaf concentrated solution prepared in Embodiment 8 in a drying oven at a constant temperature of 40-45° C. to make the water content less than 5%, then crushing, and sieving with a 100 mesh sieve to obtain mulberry leaf powder.

### Embodiment 11, Preparing of Mulberry Leaf Tablet

S1: pretreating raw and auxiliary materials: firstly, drying the mulberry leaf concentrate, okra powder, oligosaccharide and magnesium stearate prepared in Embodiment 7 in a constant temperature drying oven at 40-45° C. to make the water content less than 5%;

S2: crushing and sieving: crushing the dried mulberry leaf concentrate, okra powder, oligosaccharide and magnesium stearate respectively, and sieving the crushed raw and auxiliary materials through 100 mesh to avoid rough taste caused by large particles;

S3: mixing: mixing the mulberry leaf concentrate with okra powder in the ratio of 10:1, adding oligosaccharide accounting for 2% of the mass of mulberry leaf concentrate and 1% magnesium stearate, and mixing evenly;

S4: tablet pressing: carrying out the tablet pressing operation in a drying room with air relative humidity of 50%-60%; before and after tablet pressing, the tablet pressing part of the tablet pressing machine shall be carefully cleaned and disinfected with 75% alcohol;

S5: finished product packaging: carrying out sealed packaging under the environmental conditions of 50%-60% relative air humidity.

### Comparative Embodiment 1

The difference from Embodiment 7 is that the addition of crude cellulase enzyme solution and crude pectinase enzyme solution in S1 is omitted.

### Comparative Embodiment 2

The difference from Embodiment 8 is that the addition of crude cellulase enzyme solution and crude pectinase enzyme solution in S1 is omitted.

The functional ingredients of mulberry leaf concentrates prepared in Embodiment 7, Embodiment 8, Comparative Embodiment 1 and Comparative Embodiment 2 are detected as follows:

Main reagents and instruments

| | Reagent | |
|---|---|---|
| Reagent name | specification | Reagent manufacturer |
| Methyl alcohol | chromatographically | Tianjin Beichen Fangzheng |
| | pure | reagent factory |
| Phenol | Analytically pure | Anhui Jingcheng chemical |
| | | co., ltd |
| Concentrated sulfuric acid | Analytically pure | Tianjin Dongli District |
| | | Tianda Chemical Reagent |
| | | Factory |
| Folin phenol | Analytically pure | Hefei Bomei Biotechnology |
| | | Co., Ltd |
| Alchlor | Analytically pure | Tianjin dingshengxin |
| | | chemical co., ltd |
| Rutin | Analytically pure | Wuxi Yatai Union Chemical |
| | | Co., Ltd. |
| Glucose | Analytically pure | Chengdu Lianhe chemical |
| | | co., ltd |
| Pyrogallic acid | Analytically pure | Shanghai EBI Chemical |
| | | Reagent Co., Ltd |
| Piperidinase | Analytically pure | Hefei Bomei Biotechnology |
| | | Co., Ltd |
| Reinecke salt (Chromium | Analytically pure | Hefei Bomei Biotechnology |
| silver thiocyanate) | | Co., Ltd |
| Muriatic acid | Analytically pure | Anhui Jingcheng chemical |
| | | co., ltd |
| Ether | Analytically pure | Anhui Jingcheng chemical |
| | | co., ltd |
| Acetone | Analytically pure | Anhui Jingcheng chemical |
| | | co., ltd |
| Bovine serum albumin | Analytically pure | Beijing Solebo Technology |
| | | Co., Ltd |
| Coomassie Brilliant Blue | Analytically pure | Tianjin Dongli District |
| | | Tianda Chemical Reagent |
| | | Factory |
| Ethanol | Analytically pure | Tianjin Tianli Chemical |
| | | Reagent Co., Ltd. |
| Acetic acid | Analytically pure | Tianjin Dongli District |
| | | Tianda Chemical Reagent |
| | | Factory |
| Heat-stable α-amylase | Analytically pure | Shanghai EBI Chemical |
| solution | | Reagent Co., Ltd |
| Protease solution | Analytically pure | Shanghai EBI Chemical |
| | | Reagent Co., Ltd |
| Glucosidase solution | Analytically pure | Shanghai EBI Chemical |
| | | Reagent Co., Ltd |
| Instrument name | Model | Instrument manufacturer |
| Electronic analytical | FA224 | Shanghai Shunyu Hengping |
| balance | | Scientific Instrument Co., Ltd. |
| Digital display electronic | HH-4 | Changzhou Guohua Electrics |
| constant temperature water | | Co., ltd |
| bath pot | | |
| Rotary evaporator | XD-52A | Shanghai xiande instrument |
| | | co., ltd |
| Pipette | TopPette | Ji 'nan Dongyi Laboratory |
| | | Equipment Co., Ltd. |
| Vertical steam sterilizer | YXQ-LS-50S∥ | Shanghai boxun industry co., |
| | | ltd. medical instrument factory |
| Ultraviolet | UV-5300 | Shanghai Yuanxi Instrument |
| spectrophotometer | | Co., Ltd. |
| Sterile console | BJ-1CD | Shanghai Boxun Medical |
| | | Biological Instrument Co., Ltd |
| Acidity meter | PHS-3C | Shanghai Youke Instrument |
| | | Co., Ltd. |
| High performance liquid | Angilentl200 | Agilent Technology Co., Ltd. |
| chromatograph (HPCL) | | |
| Conventional centrifuge | SC-3612 | Anhui Zhongke Zhongjia |
| | | Scientific Instrument Co., Ltd |
| Biochemical incubator | SPC-250-∥ | Shanghai Yuejin Medical |
| | | Equipment Co., Ltd. |

Determination of moisture: determination of moisture in mulberry leaf concentrate with reference to GB 5009.3-2016 Determination of Moisture in Food.

Determination of dietary fiber: determination of soluble dietary fiber (SDF) and insoluble dietary fiber (IDF) of mulberry leaf concentrate with reference to GB 5009.88-2014 Determination of Dietary Fiber in Food.

Determination of protein: referring to SN/T39-2014 Determination of Protein Content in Exported Milk, Egg and Bean Foods by Coomassie Brilliant Blue Method to determine the protein content of mulberry leaf concentrate.

Determination of total polysaccharide: referring to NY/T1676-2008 Determination of Crude Polysaccharide Content in Edible Fungi, combined with sulfuric acid-phenol method to determine the content of polysaccharide in mulberry leaf concentrate, the mulberry leaf concentrate is treated before the determination: taking 1 mL of mulberry leaf concentrated solution, adding 30 mL of absolute ethanol, then put the solution in a refrigerator at 4° C.; taking the solution out after 48 h, centrifuging at 4000 r/min for 15 min, discarding the supernatant, washing the precipitate twice with 5 mL of 75% ethanol, dissolving in 5 mL of distilled water after drying, and putting the solution in the refrigerator at 4° C. for later use.

Determination of Total Flavonoids: referring to DB43/T 476-2009 Determination of Total Flavonoids in Foods of Plant Origin, combined with AlCl3 method to determine the content of total flavonoids in mulberry leaf concentrate. Treatment of mulberry leaf concentrate before determination: aspirating 2 mL of the concentrate, fixing the volume with anhydrous ethanol into a 50 mL volumetric flask, refrigerating at 4° C. for later use.

Determination of total polyphenols: refer to T/AHFIA 005-2018 “Determination of total polyphenols in plant extracts and their products Spectrophotometric method” combined with Folin-Denis reagent method to determine the polyphenol content in mulberry leaf concentrate. Treatment of mulberry leaf concentrate before determination: 1 mL of mulberry leaf concentrate was taken, fixed in distilled water into a 50 mL volumetric flask, refrigerated at 4° C. and set aside.

Determination of total alkaloids: taking 0.1, 0.4, 0.8, 1.2, 1.6 mL of piperidinase standard solution, fix the volume to 2 mL with 0.05 mol/L hydrochloric acid, and prepare the standard solution with the mass concentration of 0.002, 0.008, 0.016, 0.024, 0.032 mol/L, respectively. Adding 3 mL of Reinecke Salt, mixing well, ice bathing for 2 h, then centrifuging at 4000 r/min for 10 min, removing the supernatant, adding 1.0 mL of 99% ether, centrifuging at 4000 r/min for 10 min, removing the supernatant, evaporate naturally, adding 5.0 mL of 70% acetone solution to dissolve the precipitate, measuring the absorbance value at 525 nm and drawing the standard curve. Accurately weighing 2 g of the concentrate solution and adding 40 mL of 30% acidic ethanol solution (350 mL of 0.05 mol/L hydrochloric acid solution, fixed to 500 mL with anhydrous ethanol), vortex shaking for 30 s, performing ultrasonic extraction at 50° C. for 30 min, centrifuging at 5000 r/min for 15 min, collecting the supernatant, adding 40 mL of 30% acidic ethanol solution to the residue, extracting once repeatedly, combining the two extracts, concentrating under reduced pressure to a constant volume of 25 mL, adsorbing with macroporous adsorption resin HPD100 for 30 min, eluting with pure water, concentrating the eluent under reduced pressure to a constant volume of 10 mL to be tested. Taking 2 mL of mulberry leaf concentrated solution instead of piperidine enzyme standard solution, and determining the alkaloid content in mulberry leaf concentrated solution according to the above steps.

The test results are shown in Table 1:

TABLE 1

| | | Comparative | | Comparative |
|---|---|---|---|---|
| | Embodiment 7 | Embodiment 1 | Embodiment 8 | Embodiment 2 |
| Total protein mg/mL | 14.7 | 11.2 | 12.05 | 9.15 |
| Total | 6.58 | 3.7 | 3.76 | 2.43 |
| polysaccharides of | | | | |
| mulberry leaves | | | | |
| mg/mL | | | | |
| Total flavonoids of | 7.14 | 5.815 | 5.92 | 3.307 |
| mulberry leaves | | | | |
| mg/mL | | | | |
| Total polyphenol | 13.95 | 5.24 | 8.17 | 3.69 |
| of mulberry leaves | | | | |
| mg/mL | | | | |
| Total alkaloids of | 3.213 | 2.04 | 2.57 | 1.19 |
| mulberry leaves | | | | |
| mg/mL; | | | | |

It can be seen from TBL 1 that in Embodiment 7, the extraction rate of protein in mulberry leaves reaches 14.7 mg/lg, the extraction rate of polysaccharide reaches 6.58 mg/lg, the extraction rate of flavonoid reaches 7.14 mg/lg, the extraction rate of polyphenol reaches 13.95 mg/lg and the extraction rate of alkaloid reaches 3.213 mg/lg.

The functional activity of mulberry leaf concentrate prepared in Embodiment 7, Embodiment 8, Comparative Embodiment 1 and Comparative Embodiment 2 is tested, in which the calculation formula of DPPH free radical scavenging rate is as follows:

DPPH radical clearance=1−(A₁−A₂)/A₀×100%

A₁ in the formula: absorbance value after reaction between sample solution and DPPH solution; A₂: absorbance value of sample solution after reaction with absolute ethanol instead of DPPH solution; A₀: absorbance value after reaction of absolute ethanol with DPPH.

The hydroxyl radical scavenging rate is calculated as follows:

S/%=(A₀−A₁+A₂)/A₀×100

S in the formula: hydroxyl radical scavenging rate,%; A₀: absorbance value measured without sample solution; A₁: absorbance value measured by sample solution; A₂: absorbance value measured without H₂O₂.

The chelating capacity of ferrous ions is calculated as follows:

S/%=(A₀−A₁)/A₀×100

S: Fe²⁺chelating capacity,%; A₀: absorbance value measured without sample solution; A₁: absorbance value measured by sample solution.

The formula for calculating the inhibition rate of each substance on α-glucosidase is as follows:

$$Inhibition ration ( \% ) = \frac{{A}_{0} - ( {A}_{2} - {A}_{1} )}{{A}_{0}} \times 100$$

A₀: absorbance value of blank group; A₁: absorbance value of sample background group; A₂: absorbance value of sample group.

The tyrosinase inhibition rate is calculated as follows:

I (%)=1−(C-D)/(A-B)×100

A. B, C and D are the absorbance values of each solvent system in the table at 475 nm.

ACE inhibition rate is calculated by the following formula:

ACE inhibition rate (%)=(A-B)/A×100

where: A represents the absorbance value of hippuric acid generated after complete reaction between ACE and HHL; B represents the absorbance value of hippuric acid generated after the reaction of the extract with ACE and HHL; (A-B) represents the amount of hippuric acid reduced due to the addition of extract.

The test results are shown in Table 2:

TABLE 2

| | | Comparative | | Comparative |
|---|---|---|---|---|
| | Embodiment 7 | Embodiment 1 | Embodiment 8 | Embodiment 2 |
| DPPH radical | 93.47 | 78.11 | 87.19 | 77.53 |
| scavenging rate % | | | | |
| Hydroxyl radical | 90.36 | 77.51 | 83.07 | 72.8 |
| scavenging rate % | | | | |
| Chelating capacity | 84.75 | 69.5 | 75.5 | 65.1 |
| of ferrous ion % | | | | |
| α-Inhibition rate of | 88.09 | 77.98 | 82.1 | 73.21 |
| glucosidase % | | | | |
| Inhibition of | 81.9 | 69.87 | 76.3 | 60.41 |
| angiotensin | | | | |
| Tyrosinase | 78.77 | 61.69 | 66 | 53.38 |
| inhibition rate % | | | | |

It can be seen from TBL 2 that the mulberry leaf concentrate prepared by the disclosure has a DPPH radical scavenging rate of 93.47%, a hydroxyl radical scavenging rate of 90.36%, a ferrous ion chelating capacity of 84.75% α-inhibition rate of glucosidase of 88.09%, the inhibition rate of angiotensin converting enzyme of 81.9%, and the inhibition rate of tyrosinase of 78.77%.

The above described embodiments are only a description of a preferred way of the disclosure, not a limitation of the scope of the disclosure. Without departing from the spirit of the design of the disclosure, all kinds of deformations and improvements made to the technical solutions of the disclosure by a person of ordinary skill in the art shall fall within the scope of protection determined by the claims of the disclosure.

What is claimed is:

1. A method for preparing crude cellulase enzyme solution, comprising:
step 1: preparing a potato dextrose agar (PDA) slant culture medium;
step 2: preparation of cellulase-producing fermentation medium, comprising:
uniformly mixing a bran, a straw powder, an ammonium sulfate, a potassium dihydrogen phosphate, a magnesium sulfate, a calcium chloride and water, and sterilizing to obtain a cellulase-producing fermentation medium;
step 3: preparation of solid strain, comprising: inoculating a stock culture of agaric into the PDA slant culture medium for culture to obtain a mycelium;
step 4: fermentation, comprising: inoculating the mycelium into the cellulase-producing fermentation medium, uniformly mixing and culturing; and
step 5: preparation of crude cellulase enzyme solution, comprising: taking the cellulase-producing fermentation medium cultured in the step 4, adding water, shaking, standing still, filtering and centrifuging to obtain a crude cellulase enzyme solution.

2. The method for preparing crude cellulase enzyme solution according to claim 1,
wherein a pH value of a mixture of the bran, the straw powder, the ammonium sulfate, the potassium dihydrogen phosphate, the magnesium sulfate, the calcium chloride and the water uniformly mixed in the step 2 is in a range from 3.5 to 5.

3. The method for preparing crude cellulase enzyme solution according to claim 1,
wherein a temperature of the culturing in the step 4 is in a range from 25 Celsius degrees (° C.) to 32° C., and a time of the culturing is in a range from 3 days to 4 days.

4. A preparation method of mulberry leaf concentrate, comprising:
mixing mulberry leaves with water and mashing to prepare a slurry, and then adding a crude cellulase enzyme solution and a crude pectinase enzyme solution for enzymatic hydrolysis to obtain a target slurry; and
subjecting the target slurry after the enzymatic hydrolysis to enzyme killing treatment, and then concentrating to obtain a mulberry leaf concentrate;
wherein the crude cellulase enzyme solution is prepared by the method according to claim 1;
wherein the crude pectinase enzyme solution is prepared by steps of:
preparing a second PDA slant culture medium;
uniformly mixing a bran, an orange peel powder, an ammonium sulfate, a magnesium sulfate, a potassium hydrogen phosphate and water, and sterilizing to obtain a pectinase-producing fermentation medium;
taking a stock culture of Auricularia auricula into the second PDA slant culture medium for culture to obtain a second mycelium;
inoculating the second mycelium into the pectinase-producing fermentation medium, uniformly mixing and culturing to obtain a cultured pectinase-producing fermentation medium; and
taking the cultured pectinase-producing fermentation medium, adding water, shaking, standing still, filtering and centrifuging to obtain the crude pectinase enzyme solution.

5. The method for preparing mulberry leaf concentrate according to claim 4, wherein the method further comprises: filtering the slurry before the subjecting the target slurry after the enzymatic hydrolysis to enzyme killing treatment.

6. The method for preparing mulberry leaf concentrate according to claim 4, wherein a mass ratio of the crude cellulase enzyme solution to the crude pectinase enzyme solution is 3:2.

7. The method for preparing mulberry leaf concentrate according to claim 4, wherein a pH value of a mixture of the bran, the orange peel powder, the ammonium sulfate, the magnesium sulfate, the dipotassium hydrogen phosphate and the water for obtaining the pectinase-producing fermentation medium is in a range from 4 to 5.

8. The method for preparing mulberry leaf concentrate according to claim 4, wherein a temperature of the culturing to obtain the cultured pectinase-producing fermentation medium is in a range from 25° C. to 32° C., and a time of the culturing to obtain the cultured pectinase-producing fermentation medium is in a range from 3 days to 4 days.

9. A mulberry leaf product, comprising the mulberry leaf concentrate prepared by the method according to claim 4; wherein a dosage form of the mulberry leaf product is one selected from the group consisting of dropping pill, powder and tablet.
